(19) United States
(12) Patent Application Publication
(10) Pub. No.: US 2023/0098330 A1
(43) Pub. Date: Mar. 30, 2023

(54) POSTERIOR VITREOUS DETACHMENT VITRECTOMY PROBE

(71) Applicant: Alcon Inc., Fribourg (CH)

(72) Inventors: Jean-Antoine Pournaras, Lausanne (CH); Michael Cardamone, Keller, TX (US); Reto Grüebler, Greifensee (CH)

(21) Appl. No.: 17/820,643

(22) Filed: Aug. 18, 2022

Related U.S. Application Data
(60) Provisional application No. 63/250,401, filed on Sep. 30, 2021.

(51) Int. Cl. A61F 9/007

(52) U.S. Cl. CPC A61F 9/00736; A61B 2017/00858

(57) ABSTRACT

A vitrectomy probe outfitted with a vitrectomy needle having a region of enhanced frictional character which may be referred to as an “augmented” surface region. This region may be utilized to interface with tissues such as the hyaloid membrane for forcibly shearing it from, for example, the underlying retina in a manner that avoids a degree of pulling forces on the retina in achieving the hyaloid detachment. By utilizing the vitrectomy probe to achieve the detachment, a subsequent intervention to introduce the probe for uptake of the sheared or detached hyaloid membrane material may also be avoided.

### PRIORITY CLAIM

This application claims the benefit of priority of U.S. Provisional Patent Application Ser. No. 63/250,401 titled “POSTERIOR VITREOUS DETACHMENT VITRECTOMY PROBE,” filed on Sep. 30, 2021, whose inventors are Jean-Antoine Pournaras, Michael Sam Cardamone and Reto Grüebler, which is hereby incorporated by reference in its entirety as though fully and completely set forth herein.

### BACKGROUND

Over the years, many dramatic advancements in the field of eye surgery have taken place. One of the more common eye surgery procedures is a vitrectomy. Vitrectomy is the removal of some or all of the vitreous humor from a patient's eye. In some cases, where the surgery is limited to removal of clouded vitreous humor, the vitrectomy may constitute the majority of the procedure. However, a vitrectomy may accompany cataract surgery, surgery to repair a retina, to address a macular pucker or a host of other issues.

One of the procedures that often accompanies a vitrectomy and/or is facilitated by the use of a vitrectomy probe is a posterior vitreous detachment (PVD). That is, a vitreous membrane is generally present serving as a border between the vitreous humor and the retina. At the back of the eye, this membrane of fibrous film may be referred to as the posterior hyaloid membrane and may be considered a “false membrane”. Removal of this membrane may be intentionally done to address distorted vision or as a precautionary measure to avoid the more serious event of a retinal detachment as described below.

Generally, as people age, there is an increased tendency for the hyaloid to separate or slightly detach from the underlying sensitive and more critical retina. When this occurs, symptoms such as flashes of light and/or an increase in the presence of “floaters” may emerge. More seriously though, this separation may be a precursor to retinal detachment.

A retinal detachment may present with some of the same issues as a detached hyaloid as described above. However, a retinal detachment may also include much more severe vision complications. Blurred vision, the inability to see in dim light, tunnel vision and even loss of vision altogether may occur with a retinal detachment. As a result, efforts to prevent retinal detachment may be undertaken following the presentation of a hyaloid detachment.

One such preventative measure that may be undertaken is to remove the separating hyaloid. By eliminating the separating hyaloid membrane with a PVD procedure, a pull by the membrane on the underlying retina is also eliminated. Thus, a subsequent retinal detachment may be avoided. Of course, even where a retinal detachment has occurred, a PVD procedure may be undertaken as part of the repair. Furthermore, even where a hyaloid detachment has not begun, there may be circumstances in which a PVD may be undertaken, such as a preventative measure in conjunction with another eye procedure that is already being undertaken. Regardless, PVD procedures have become fairly commonplace over the years.

As with other eye surgeries, a PVD procedure may involve a fairly standard set of minimally invasive techniques. For example, small pre-placed cannulas at offset eye locations may be positioned to support and guide interventional instruments into the eye. This may include a light instrument, the above noted vitrectomy probe and even a pick-like instrument for performing the PVD itself.

Of course, with the limited workspace surrounding the eye and the surgeon only having two hands, rather than make three or more incisions and cannula placements, the PVD may take place followed by the vitrectomy through the same cannula location. That is, the PVD instrument may first be used to complete the intentional PVD separation, for example, in younger patients where the membrane is more securely attached to the retina below. Subsequently, the PVD instrument may be removed and the vitrectomy probe introduced to achieve the cutting and uptake of the, now floating, hyaloid membrane and other vitreous humor.

Unfortunately, the described sequence and techniques present potential hazards. For example, the separation of the hyaloid membrane from the underlying retina by way of the PVD instrument may place a pulling force on the retina. Thus, the risk of retinal detachment and potential loss of eyesight or other eye problems is presented. Additionally, there is often the removal of one surgical PVD instrument followed by the insertion of another vitrectomy tool. That is multiple interventions, each with their own inherent risks may be required in order to complete the PVD procedure.

### SUMMARY

Several instruments may benefit from the addition of an augmentation region on an outer surface. For example, a vitrectomy probe (which includes a handpiece held by a surgeon having a vitrectomy needle emerging therefrom) may benefit from an augmentation region on an outer surface of the needle (e.g., near a port which uptakes, for example, hyaloid material from the eye). The augmentation region may include a surface with an enhanced frictional character for dislodging of, for example, the hyaloid material from the underlying retina.

Other examples of instruments that may also include an augmentation region include, for example, an endoilluminator probe, a scissors, a forceps, a membrane pik, a delamination spatula, a macular lens, an aspiration handpiece, a membrane scraper, and a laser probe. Furthermore, various locations on the instruments (e.g., as outlined below) may be used.

### BRIEF DESCRIPTION OF THE DRAWINGS

FIG. 1 is a side perspective view of a vitrectomy probe with an embodiment of a vitrectomy needle having an augmented surface to facilitate an eye procedure.

FIGS. 2A-C are enlarged partially cross-sectional views of a hyaloid membrane in an eye of a patient being lifted by the needle of FIG. 1 with the augmented surface on various portions of the vitrectomy probe.

FIG. 2D illustrates a side sectional view of a vitrectomy probe having the augmented surface on a side and beveled surface.

FIG. 2E illustrates a side sectional view of a vitrectomy probe having the augmented surface on a rounded tip between the bevel and the front surface.

FIG. 3 is a side sectional overview of the probe of FIG. 1 being utilized in an eye procedure, including at the membrane of FIGS. 2A-C.

FIG. 4A is a view of the augmented surface of the needle region of FIG. 1 revealing individual projection rows thereat.

FIG. 4B is another view of the augmented surface of the needle region revealing individual projections forming the rows of FIG. 4A.

FIG. 5 is a flow-chart summarizing an embodiment of utilizing a vitrectomy probe needle with an augmented surface to facilitate an eye procedure (e.g., a PVD procedure).

FIG. 6 illustrates a straight endoilluminator probe having the augmented surface on a distal portion of the probe.

FIG. 7a illustrates a curved scissors having the augmented surface on an inner surface of the scissors.

FIG. 7b illustrates a curved scissors having the augmented surface on an outer surface of the scissors.

FIG. 8 illustrates a straight scissors having the augmented surface on a distal portion of the scissors.

FIG. 9a illustrates a vertical scissors having the augmented surface on an internal surface of the scissors.

FIG. 9b illustrates a vertical scissors having the augmented surface on an outer curved surface of the scissors.

FIG. 10 illustrates a triangular forceps (e.g., GRIESHABER MAXGRIP® forceps) having the augmented surface on a distal portion of the forceps.

FIG. 11 illustrates an end grasping forceps having the augmented surface on a distal portion of the forceps.

FIG. 12 illustrates an asymmetrical forceps having the augmented surface on a distal portion of the forceps.

FIG. 13 illustrates a serrated forceps having the augmented surface on a distal portion of the forceps.

FIG. 14 illustrates a membrane pik having the augmented surface on a distal portion of the pik.

FIG. 15 illustrates an illuminated membrane pik having the augmented surface on a distal portion of the pik.

FIG. 16 illustrates a delamination spatula having the augmented surface on a distal portion of the spatula.

FIG. 17 illustrates a flexible membrane scraper having the augmented surface on a distal portion of the scraper.

FIG. 18a illustrates a straight laser probe having the augmented surface on a distal portion of the probe.

FIG. 18b illustrates a curved laser probe having the augmented surface on a distal portion of the probe.

FIG. 19 illustrates a macular lens with tabs having the augmented surface on a bottom surface of the tabs.

FIG. 20 illustrates an aspiration handpiece having the augmented surface on a distal portion of the tip.

### DETAILED DESCRIPTION

In the following description, numerous details are set forth to provide an understanding of the present disclosure. However, it will be understood by those skilled in the art that the embodiments described may be practiced without these particular details. Further, numerous variations or modifications may be employed which remain contemplated by the embodiments as specifically described.

Embodiments are described with reference to certain types of eye procedures (e.g., PVD and vitrectomy surgical procedures). While embodiments described herein describe a procedure in which a PVD is directly and uniquely performed with a vitrectomy probe, it is to be understood that the modified vitrectomy probe described and claimed herein may be used in other types of surgical procedures (e.g., tractional retinal detachment (TRD), etc. or other procedures that may benefit from both cutting and tissue manipulation with a single device). In some embodiments, the probe may continue to be utilized for the uptake of the posterior vitreous membrane or hyaloid and even continue to be utilized, for example to address a vitreous hemorrhage or other eye issues. Of course, a variety of other maneuvers may be carried out with the probe over the course of a single intervention. Regardless, so long as an augmented surface region of the vitrectomy needle is utilized to facilitate, for example, a PVD procedure, appreciable benefit may be realized.

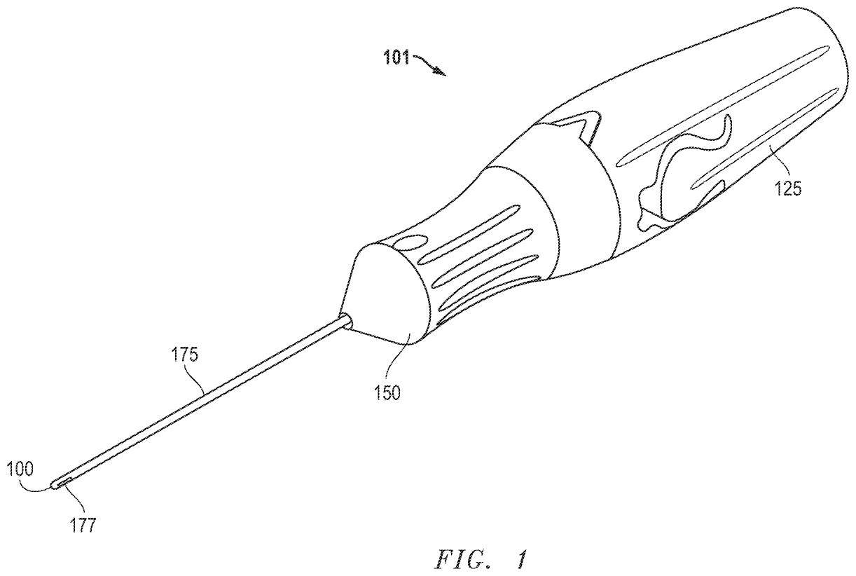

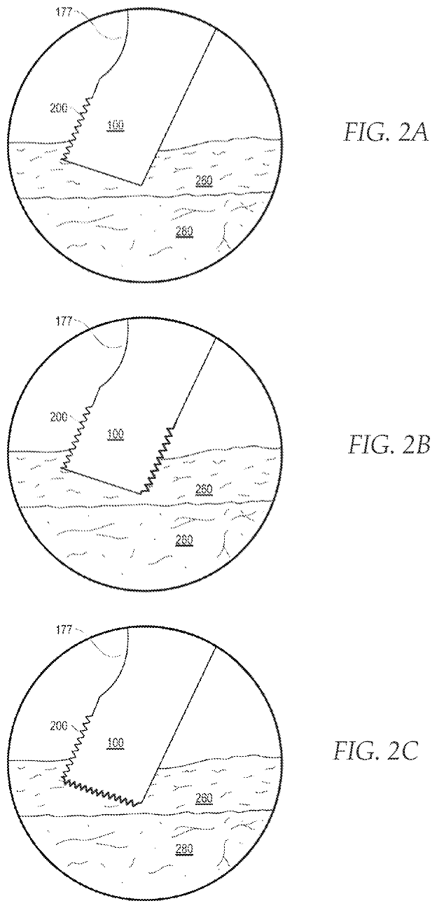

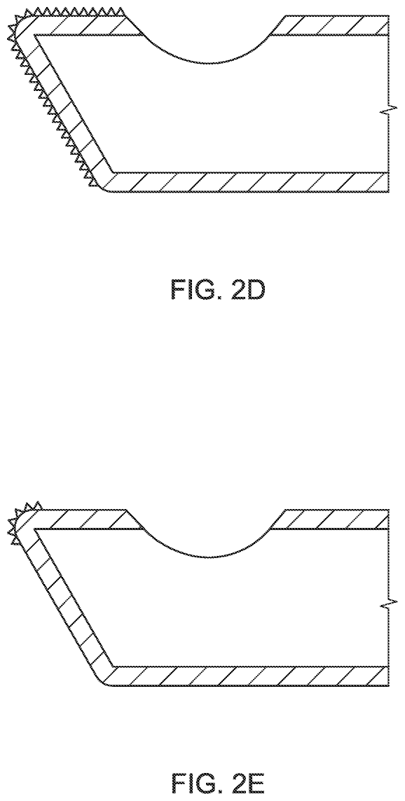

Referring now to FIG. 1, a side perspective view of a vitrectomy probe 101 is illustrated. The probe 101 is outfitted with an embodiment of a vitrectomy needle 175 having an augmented surface region 100 to facilitate, for example, a PVD procedure. This is well illustrated in FIGS. 2A-C where an enlarged view of the region 100 is depicted with teeth 200 that are used to physically interface, for example, the hyaloid membrane 260 to shearingly facilitate detachment of the membrane from the underlying retina 280 as detailed below. The augmented surface region 100 may be referred to as a laser structure or sharkskin region due to the mode of manufacture and/or the frictional feel at its surface.

Continuing with reference to FIG. 1, the vitrectomy probe 101 may otherwise be of standard convention. In the embodiment shown, the handpiece portion of the probe 101 includes a component housing 150 which accommodates mechanical features directed at reciprocating a cutter within the needle 175. Thus, vitreous humor, detached hyaloid material or other substances may be taken up into the port 177 and transported out of the eye of the patient.

The component housing 150 of FIG. 1 includes a tapered end for holding by a surgeon, generally between a thumb and forefinger during use of the probe 101. In the embodiment shown, a removable shell 125 is also provided at the proximal end of the housing 150. Thus, the probe 101 may be supported at the purlicue between the thumb and forefinger if the surgeon so chooses. Of course, the probe may be outfitted with a variety of other supportive features and aspects.

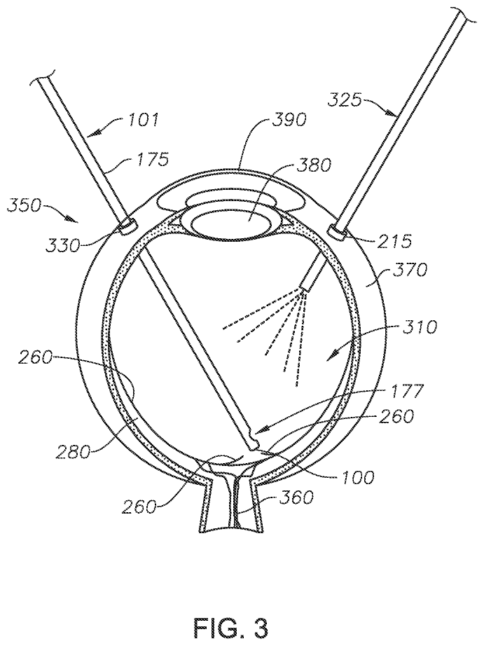

Referring to FIGS. 2A-C, enlarged partially cross-sectional views of a hyaloid membrane 260 are shown with the augmented surface 100 on various portions of the vitrectomy probe. With added reference to FIG. 3, this membrane 260 is found within an eye 350 of a patient and on a patient's retina 280. For various reasons, a PVD procedure to remove the hyaloid membrane 260 may be sought as noted herein. As illustrated, the probe needle 175 may be directly manipulated by a surgeon to lightly but forcibly interface the augmented surface region 100 thereof with the membrane 260. In this way, frictional shearing forces may be imparted through teeth 200 of the region 100 onto the membrane 260. This is in contrast to pulling or other forces that might alternatively be employed in a conventional PVD. With added reference to FIG. 3, to the extent that such shearing forces are imparted to break up and/or detach the hyaloid membrane 260, pulling forces prone to pull on and potentially damage the underlying retina 280 may be avoided.

In certain circumstances, the hyaloid membrane 260 may be particularly adherent to the underlying retina 280, for example, in younger patients or at locations closer to the vitreous base and optic nerve. Thus, the avoidance of pulling on the membrane 260 during the PVD may be of healthy benefit to the retina 280. Furthermore, the hyaloid membrane 260 is a film of fibrous deposit, generally considered to be a false membrane. Once it is detached in the PVD procedure it may be taken up and cut up through the adjacent port 177. Thus, there is no concern about shearing, tearing or cutting the hyaloid membrane 260 into various chunks or particulate as opposed to a more uniform conventional lifting or pulling. In some circumstances, the augmented region 100 may even be slightly embedded into the membrane 260 or even into a space between this membrane 260 and the retina 280 in advance of shearing.

In the embodiment of FIGS. 2A-C, the manner in which the augmented region 100 interfaces the hyaloid membrane 260 may be tailored to the type of maneuvering likely to take place by the surgeon in imparting the shearing forces. For example, the surgeon might be expected to forcibly move the end of the needle 175 forward or backward (to the left or right as illustrated). Thus, the augmented region 100 may be arranged into horizontal rows of individual teeth 200 that would be arranged largely perpendicular to the noted forward or backward motion. Of course, a variety of alternative morphologies or architectures may also be employed. As see in FIGS. 2A-C, in some embodiments, the augmented region 100 may be placed between the port 177 and the distal end of the probe 100. The augmented region 100 may be formed as a patch that partially (e.g., as shown in FIG. 2A) or fully circumscribes (as shown in FIG. 2B) the portion of the probe adjacent the distal end. In some embodiments, the augmented region 100 may be formed on the distal end of the probe (as seen in FIG. 2C). In some embodiments, the augmented region 100 may be formed on the sides and end of the probe. In another example, the augmented surface may be placed on a side and beveled surface of a vitrectomy probe (e.g., as seen in FIG. 2D). As yet another example, the augmented surface may be placed on a rounded tip of the vitrectomy probe between the bevel and the front surface (e.g., as shown in FIG. 2E).

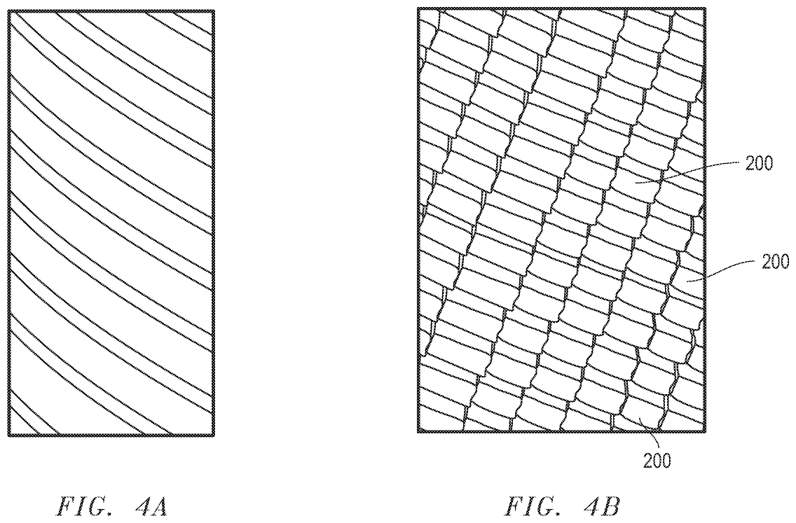

For example, the teeth 200 may be continuous ribs as illustrated in FIG. 4A or made up of rows of individual scales as illustrated in FIG. 4B, presenting a frictional grid to the adjacent tissue membrane 260, in either case. Once more, the depicted teeth 200 emerge perpendicularly or horizontally from the needle 175 for a distance of between about 3 and 40 microns in height or profile. However, this is not required and in one embodiment, the teeth 200 may be angled in the forward or backward direction such that movement in such direction encourages shearing of the hyaloid membrane 260. Indeed angling in the backward direction is illustrated in FIGS. 2A-C. For perspective, with the needle 175 perpendicular the membrane 260, this angling may be anywhere between about 10° and 90°.

Referring now to FIG. 3, a side sectional overview of the probe 101 of FIG. 1 being utilized in a PVD procedure in the eye 350 is illustrated. In particular, the hyaloid membrane 260 is shown as it is being shearingly detached from the underlying retina 260. During this surgical procedure, the vitrectomy probe 101 is utilized to frictionally interface the augmented region 100 with the membrane 260 to facilitate the depicted detachment. Of course, this begins with the needle 175 of the probe 101 being inserted through a preplaced cannula 330 and directed toward the anterior region of the eye 350.

Once the hyaloid membrane 260 is fully detached, a suction is applied and the port 177 of the probe 101 may be utilized for the uptake of detached hyaloid material. Further, with the vitrectomy probe 101 in place as illustrated, conventional vitrectomy directed at the uptake of vitreous humor or other substances may take place with the same single intervention. For example, in the procedure illustrated, a hemorrhage may be taking place in the region 310 such that blood is also drawn into the port 177 along with the vitreous humor, and preceding hyaloid material. Of course, this region may include hyaloid material that presented as floaters in advance of the PVD procedure. Regardless, the same vitrectomy may be used for the uptake of such material.

Recall that a cutter reciprocating within the needle 175 during this delicate procedure helps to ensure that the uptake of these biological materials occurs in a manner that helps avoid damage to adjacent eye features. Further, as illustrated, the surgery includes the probe 101 and a light instrument 325 reaching into the eye 350 through cannulas 315, 330 positioned in an offset manner at the sclera 370. In this way, the more delicate cornea 390 and lens 380 may be avoided. By the same token, the optic nerve 360 and retina 280 are also quite delicate. Therefore, the use of a shearing or frictional probe technique for detaching the hyaloid membrane 260 during PVD may be of substantial benefit.

Referring now to FIGS. 4A and 4B, closer views of embodiments of the augmented surface region 100 are shown such as might be apparent via a scanning electron microscope (SEM). Specifically, FIG. 4A illustrates how teeth 200 in rows as illustrated in FIGS. 2A-C might appear. Alternatively, FIG. 4B illustrates individual teeth 200 as referred to above which might present a frictional grid surface to adjacent hyaloid membrane 260 as shown in FIGS. 2A-C and 3. In the embodiment of FIG. 4B, these projections 200 are roughly 15×15 micron with about a 3-50 micron depth. Other dimensions are also contemplated (e.g., 10×10 microns, 20×20 microns, 25×25 microns, etc.) Once more, the teeth 200 may take on a variety of different shapes such as four-sided pyramidal, cylindrical, circular, polygonal, rectangular, square, scalloped or a host of other morphologies. Further, notice that although presented as individual teeth, the projections 200 are aligned in rows similar to that of FIG. 4A.

Referring now to FIG. 5, a flow-chart summarizing an embodiment of utilizing a vitrectomy probe needle with an augmented surface to facilitate a PVD procedure is illustrated. Specifically, the needle is advanced toward a hyaloid membrane in an eye of a patient as indicated at 515. The needle and augmented surface may be used to achieve a shearing type of detachment of the hyaloid from the underlying retina (see 540). As indicated at 540, the detached hyaloid material may be taken up by the needle already present for the PVD. Indeed, as noted at 590, the needle may be employed to facilitate a vitrectomy while still in the eye.

Embodiments described hereinabove include the use of a vitrectomy probe to address and facilitate a PVD procedure by way of a needle implement having an enhanced frictional character. Thus, to a large extent, peeling or pulling forces to lift and detach the hyaloid membrane from the underlying retina may be avoided. As a result, the tendency to unintentionally detach or damage the retina with pulling forces may also be largely avoided. Furthermore, since the same instrument is being utilized for the PVD that is being used for subsequent uptake of the hyaloid and other vitrectomy tasks, the number of interventions may be beneficially reduced.

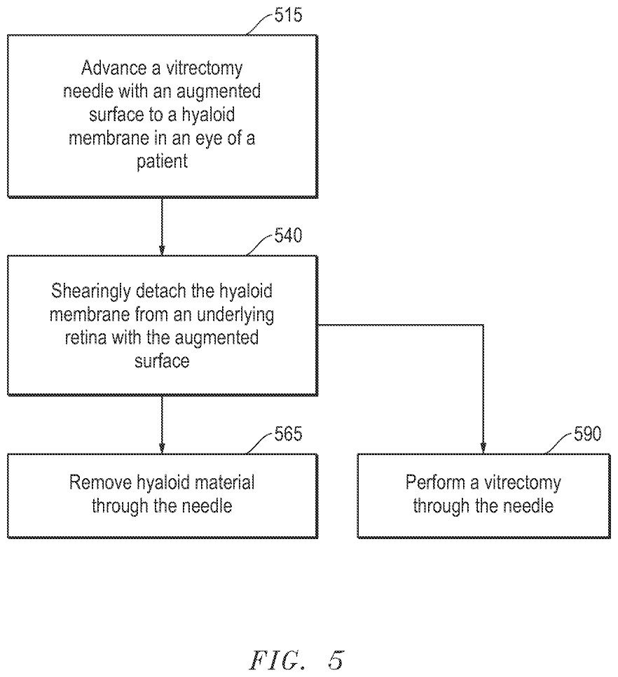

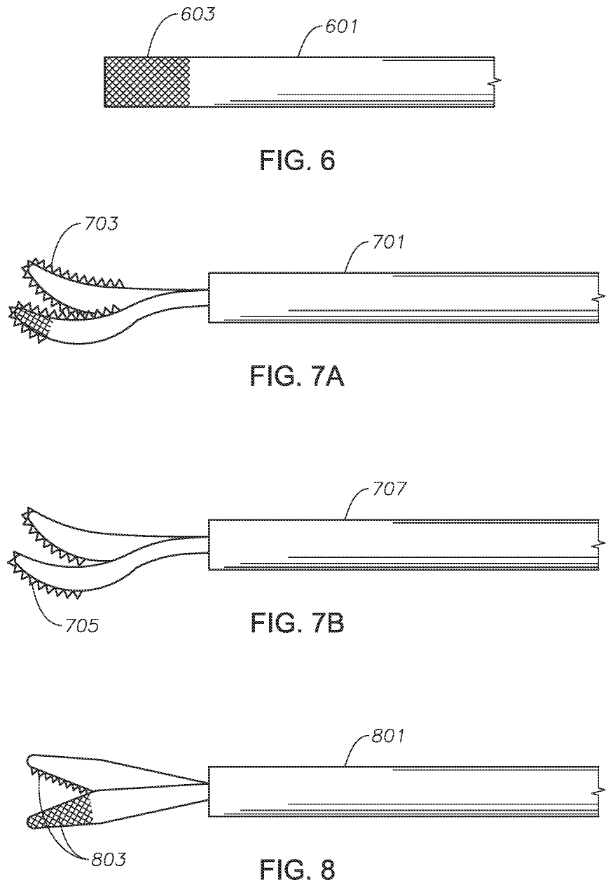

FIG. 6 illustrates a straight endoilluminator probe 601 having the augmented surface 603 on a distal portion of the probe 601. The augmented surface 603 may be placed around the entire probe distal end or on a portion (e.g., through 180 degrees of circumference around the probe distal end). Other portions (e.g., 90 degrees, 45 degrees, etc.) are also contemplated. In some embodiments, the augmented surface 603 may be placed only near a tip of the probe distal end.

In some embodiments, the augmented surface may be placed on scissors (e.g., augmented surface 703 on an inner surface of curved scissors 701 in FIG. 7a). As another example, augmented surface 705 may be used on an outer surface of curved scissors 707 in FIG. 7b. In some embodiments, the augmented surface may be placed along the cutting edge of the scissors.

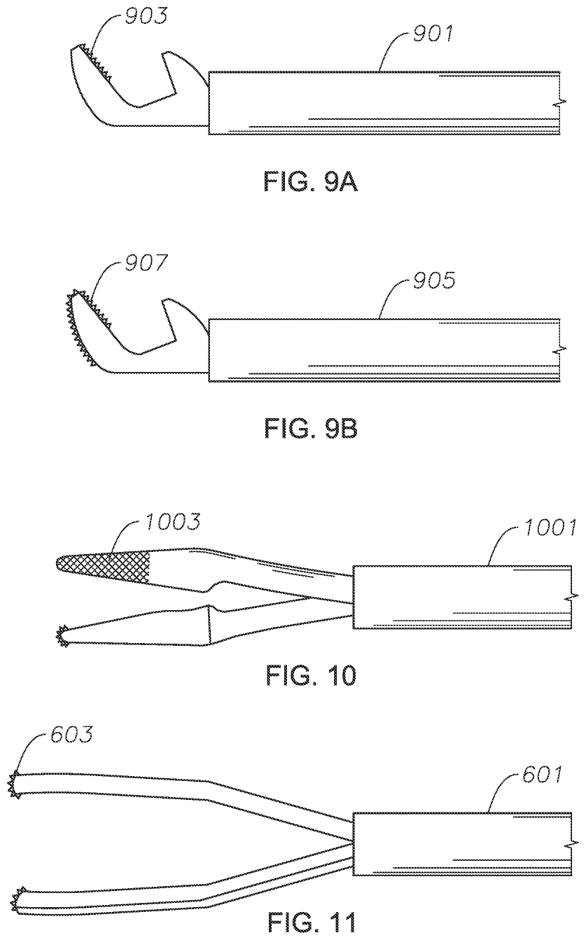

The augmented surface may be used on other types of scissors as well. For example, as seen in FIG. 8, the augmented surface 803 may be used on a distal portion of straight scissors 801. As yet another example, the augmented surface 903 may be used on an internal surface of vertical scissors 901 as seen in FIG. 9a or on an outer curved surface of vertical scissors 905 (see augmented surface 907 in FIG. 9b).

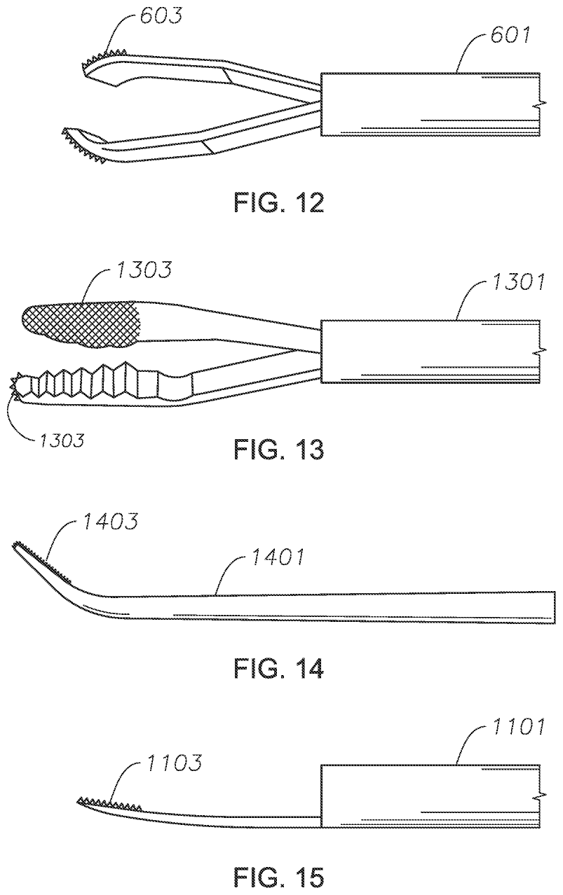

In some embodiments, the augmented surface 1003 may be used on forceps. For example, FIG. 10 illustrates a triangular forceps 1001 (e.g., GRIESHABER MAXGRIP® forceps) having the augmented surface 1003 on a distal portion of the forceps 1001. FIG. 11 illustrates another example of an augmented surface 1103 on a distal portion of an end grasping forceps 1101. As yet another example, FIG. 12 illustrates an asymmetrical forceps 1201 having the augmented surface 1203 on a distal portion of the forceps. Further, FIG. 13 illustrates a serrated forceps 1301 having the augmented surface 1303 on a distal portion of the forceps 1301. In some embodiments, the augmented surface may be placed on the grasping platform of the forceps (e.g., to a height approximately in a range of one to five microns).

The augmented surface may further be used on a membrane pik (e.g., see FIG. 14 showing the augmented surface 1403 on a distal portion of the pik 1401 and FIG. 15 showing an illuminated membrane pik 1501 having the augmented surface 1503 on a distal portion of the pik 1501).

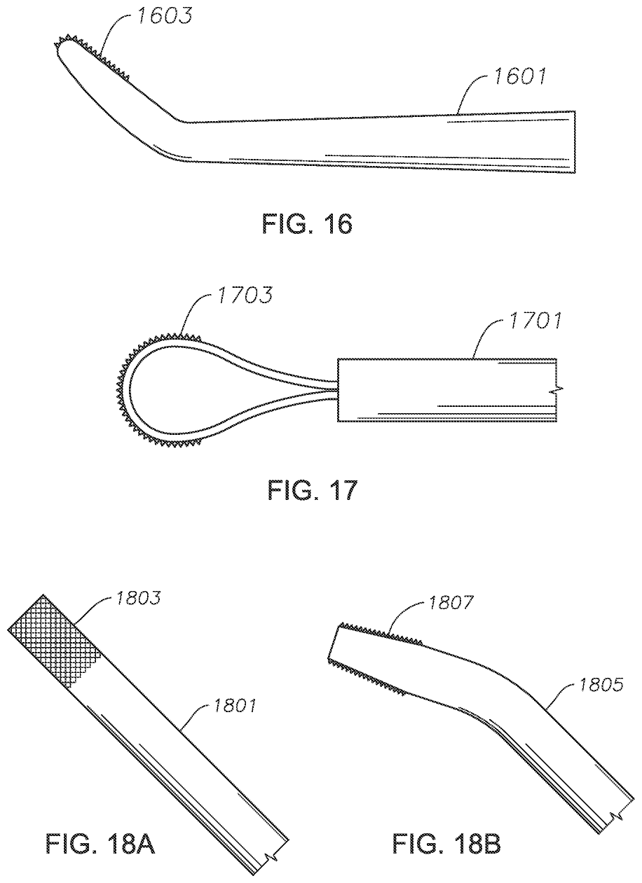

FIG. 16 illustrates a delamination spatula 1601 having the augmented surface 1603 on a distal portion of the spatula 1601. In some embodiments, the augmented surface 1603 may be on an inside surface of the spatula 1601. In some embodiments, the augmented surface 1603 may be on the outer curve of the spatula 1601.

FIG. 17 illustrates a flexible membrane scraper 1701 having the augmented surface 1703 on a distal portion of the scraper 1701. In some embodiments, the augmented surface 1703 may be on an outer curve of the scraper 1701. The augmented surface 1703 may be on a distal most portion of the scraper 1701 and may, in some embodiments, extend at least partially down the side the loop of the scraper 1701. In some embodiments, the entire loop may include the augmented surface 1703.

FIG. 18a illustrates a straight laser probe 1801 having the augmented surface 1803 on a distal portion of the probe 1801. In some embodiments the laser probe 1801 may include the augmented surface 1803 on a distal side portion of the laser probe 1801. In some embodiments, the augmented surface 1803 may extend all the way around the distal sides of the probe 1801. In some embodiments, a smaller angle of coverage may be used (e.g., 180 degrees, 90 degrees, 45 degrees, etc.)

FIG. 18b illustrates a curved laser probe 1805 having the augmented surface 1807 on a distal portion of the probe 1805. In some embodiments, the augmented surface 1807 may be placed on an inside and/or outside curve of the laser probe 1805. In some embodiments, the augmented surface 1807 may extend around the entire circumference of the distal end of the laser probe 1805. In some embodiments, a smaller angle of coverage may be used (e.g., 180 degrees, 90 degrees, 45 degrees, etc.)

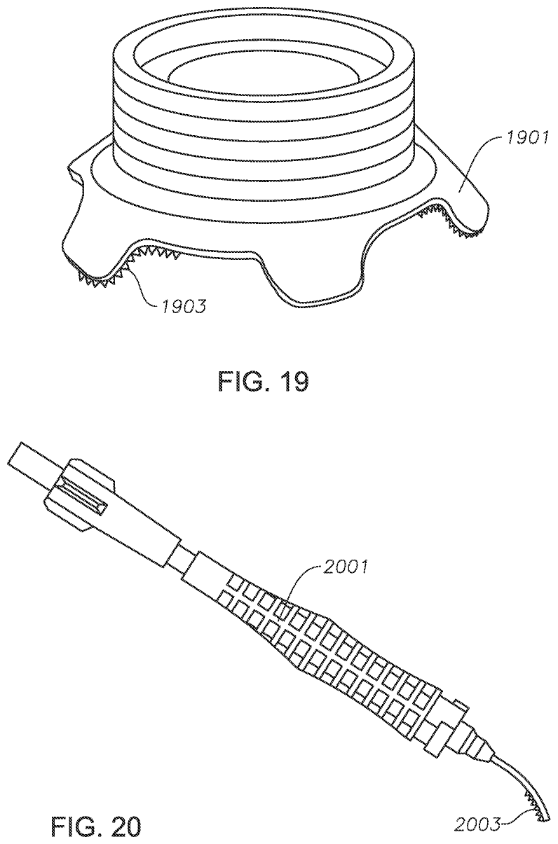

FIG. 19 illustrates a macular lens 1901 with tabs having the augmented surface 1903 on a bottom surface of the tabs. In some embodiments, a bottom surface of the tabs (the surface that comes in contact with the eye) may include the augmented surface 1903. In some embodiments, the augmented surface 1903 may be on all of the tabs. In some embodiments, the augmented surface 1903 may be on a subset of the tabs (e.g., every other tab).

FIG. 20 illustrates an aspiration handpiece 2001 having the augmented surface 2003 on a distal portion of the tip. In some embodiments, the augmented surface 2003 may be placed on an inner curve of a tip of the aspiration handpiece 2001. In some embodiments, the augmented surface 2003 may be placed on an outer surface of the aspiration handpiece 2001. In some embodiments, the augmented surface 2003 may extend around the entire circumference of the distal end of the aspiration handpiece 2001. In some embodiments, a smaller angle of coverage may be used (e.g., 180 degrees, 90 degrees, 45 degrees, etc.)

The preceding description has been presented with reference to some embodiments. However, other embodiments and/or features of the embodiments disclosed but not detailed hereinabove may be employed. Furthermore, persons skilled in the art and technology to which these embodiments pertain will appreciate that still other alterations and changes in the described structures and methods of operation may be practiced without meaningfully departing from the principle and scope of these embodiments. Additionally, the foregoing description should not be read as pertaining only to the precise structures described and shown in the accompanying drawings, but rather should be read as consistent with and as support for the following claims, which are to have their fullest and fairest scope.

1. A vitrectomy probe, comprising:
a handpiece;
a vitrectomy needle secured to the handpiece and accommodating a port for uptake of vitreous or hyaloid material from the eye; and
an augmentation region at an outer surface of the needle with an enhanced frictional character for engaging tissue to facilitate dislodging or uptake thereof.

2. The vitrectomy probe of claim 1 wherein the augmentation region comprises teeth to provide the enhanced frictional character.

3. The vitrectomy probe of claim 2 wherein the teeth are arranged in one of continuous ribs and individual scales.

4. The vitrectomy probe of claim 3 wherein the one of the continuous ribs and individual scales are further arranged in horizontal rows.

5. A needle for a vitrectomy probe, comprising:
an outer surface defining a port for uptake of vitreous or a hyaloid membrane of an eye; and
an augmentation region at an outer surface of the needle with an enhanced frictional character to engage tissue to facilitate dislodging or uptake thereof.

6. The needle of claim 5 wherein the augmentation region comprises teeth to provide the enhanced frictional character.

7. The needle of claim 6 wherein the teeth are arranged into horizontal rows.

8. The needle of claim 6 wherein with the needle oriented perpendicular to a hyaloid membrane, the teeth are angled at between about 10° and 90° thereto.

9. The needle of claim 6 wherein the teeth are of a profile of between about 3 and about 50 microns.

10. The needle of claim 6 wherein the teeth are arranged in one of continuous ribs and individual scales of about 10 by 10 microns to 25 by 25 microns.

11. The needle of claim 10 wherein each of the individual scales are of a shape selected from a group consisting of four-sided pyramidal, cylindrical, circular, polygonal, rectangular, square and scalloped.

12. A method, the method comprising:
advancing a vitrectomy needle through a pre-placed cannula at the eye and toward the membrane;
interfacing an augmentation region at an outer surface of the needle with the membrane; and
moving the region during the interfacing for detaching the at least a portion of the membrane from a retina of the eye therebelow.

13. The method of claim 12 wherein the detaching comprises shearing of the at least a portion of the membrane from the retina.

14. The method of claim 12 further comprising removing the at least a portion of the membrane through a port in the needle adjacent the region.

15. The method of claim 14 further comprising performing a vitrectomy with the needle remaining through the pre-placed cannula.
